(19) United States
(12) Patent Application Publication
(10) Pub. No.: US 2022/0111289 A1
(43) Pub. Date: Apr. 14, 2022

(54) REVOLUTIONZ THE ULTIMATE SHOE

(71) Applicant: Eddie James WILLIAMS, Goodyear, AZ (US)

(72) Inventor: Eddie James WILLIAMS, Goodyear, AZ (US)

(21) Appl. No.: 17/561,864

(22) Filed: Dec. 24, 2021

Related U.S. Application Data
(63) Continuation-in-part of application No. 14/810,076, filed on Jul. 27, 2015.
(60) Provisional application No. 62/114,946, filed on Feb. 11, 2015.

(51) Int. Cl. A63F 13/212; A63B 24/00; A63F 13/218; A63F 13/327; A63F 13/816; A63F 13/285

(52) U.S. Cl. CPC A63F 13/212; A63B 24/0062; A63F 13/218; H04R 1/1016; A63F 13/816; A63F 13/285; A63F 13/327

(57) ABSTRACT

The invention is of a workout system with a pair of shoes, a handheld device, and wireless transformable game controller headphones. The pair of shoes, the handheld device, and the wireless transformable game controller headphones are communicatively coupled. Force feedback, vibrational feedback, a shake, a jolt, and other interactive motion are emitted by the devices. The wireless transceiver of the handheld device exchanges data with the wireless transceiver of the pair of shoes and the wireless transformable game controller headphones.

### CROSS-REFERENCE TO RELATED APPLICATION

This application claims the benefit of priority of U.S. provisional application number 62/114,946, filed Feb. 11, 2015, the contents of which are herein incorporated by reference.

This application is a continuation in part of nonprovisional application number 14,81,0076, filed Jul. 27, 2015, the contents of which are herein incorporated by reference.

This application is a continuation in part of nonprovisional application number 14,81,0076, filed Oct. 12, 2021, the contents of which are herein incorporated by reference.

### BACKGROUND OF THE INVENTION

The present invention relates to a workout system and, more particularly, to a workout system including an electronic chip within a pair of shoes linked with a handheld device.

Physical exercise is any bodily activity that enhances or maintains physical fitness and overall health and wellness. It is performed for various reasons, including strengthening muscles and the cardiovascular system, honing athletic skills, weight loss or maintenance, and merely enjoyment. Frequent and regular physical exercise boosts the immune system and helps prevent the “disease of affluence” such as heart disease, cardiovascular disease, Type 2 diabetes, and obesity. It may also help prevent depression, help to promote or maintain positive self-esteem, improve mental health generally, and can augment an individual's sex appeal or body image, which has been found to be linked with higher levels of self-esteem.

As can be seen, there is a need for interactive devices that may be used for exercise.

### SUMMARY OF THE INVENTION

In one aspect of the present invention, a workout system comprises: a pair of shoes, wherein at least one of the pair of shoes comprises an electronic chip comprising an impact detector and a wireless transceiver; and a handheld device comprising a processor, a wireless transceiver, a global positioning system, and a monitor, wherein the wireless transceiver of the handheld device exchanges data with the wireless transceiver of the pair of shoes wherein the monitor displays at least one of an amount of impacts detected by the impact detector, a location of the handheld device, a distance traveled of the handheld device, and a speed of the handheld device.

These and other features, aspects and advantages of the present invention will become better understood with reference to the following drawings, description and claims.

### BRIEF DESCRIPTION OF THE DRAWINGS

FIG. 1 is a schematic view of an embodiment of the present invention.

FIG. 2 is a flow chart of the general use of the present invention.

FIG. 3 is a schematic view of the monitor of FIG. 1 displaying a GPS mode.

FIG. 4 is a flow chart of the GPS function of FIG. 3.

FIG. 5 is a schematic view of the monitor of FIG. 1 displaying a music mode.

FIG. 6 is a flow chart of the music function of FIG. 6.

FIG. 7 is a schematic view of the monitor of FIG. 1 displaying safety lights mode.

FIG. 8 is a flow chart of the safety lights function of FIG. 7.

FIG. 9 is a schematic view of the monitor of FIG. 1 displaying workout programs mode.

FIG. 10 is a flow chart of the workout programs function of FIG. 9.

FIG. 11 is a schematic view of the monitor of FIG. 1 displaying distress signal mode.

FIG. 12 is a flow chart of the distress signal function of FIG. 11.

FIG. 13 is a schematic view of the monitor of FIG. 1 displaying biking mode.

FIG. 14 is a flow chart of the biking functions of FIG. 13.

FIG. 15 is a schematic view of the monitor of FIG. 1 displaying texting mode.

FIG. 16 is a flow chart of the texting functions of FIG. 15.

FIG. 17 is a schematic view of the monitor of FIG. 1 displayingapplication store mode.

FIG. 18 is a flow chart of the application store function of FIG. 17.

FIG. 19 is a schematic view of the monitor of FIG. 1 displaying heart rate mode.

FIG. 20 is a flow chart of the heart rate function of FIG. 19.

FIG. 21 is an exemplary flow chart of the force feedback function.

FIG. 22 is an exemplary flow chart of the force feedback function.

FIG. 23 is an exemplary embodiment of the wireless transformable game controller.

FIG. 24 is an exemplary embodiment of the detachable member of the wireless transformable game controller.

FIG. 25 is an exemplary embodiment of the controllers of the wireless transformable game controller.

FIG. 26 is an exemplary embodiment of the pair of shoes of the present invention.

FIG. 27 is an exemplary embodiment of the wireless earbuds of the present invention.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims. Broadly, an embodiment of the present invention provides a workout system including athletic shoes equipped with lights powered by batteries for safe night time running, and an electronic chip that contains information that may be fed into a hand held monitor. The monitor may control the lights and can transmit music or other information to wireless ear buds for the exerciser's enjoyment.

The electronic chip and the night lights may be built inside of the shoe. The hand held monitor allows viewing the exercisers' workout progress by time and distance. The present invention may include a Global Position System (GPS), night time workouts, and the exerciser may download and listen to music with the wireless ear buds. The shoe transmits the information and saves it to the monitor. The features may be activated from the workout monitor.

The system has batteries inside the shoe and has an informational chip inside of the soul. The chip may transmit the information to the monitor. The monitor has the GPS system and workout programs. The monitor also may include enough memory to save workout progress and download music. To use the present invention, the exerciser turns on the monitor, puts on the shoes, calibrates the GPS system and sets the features the user would like to use.

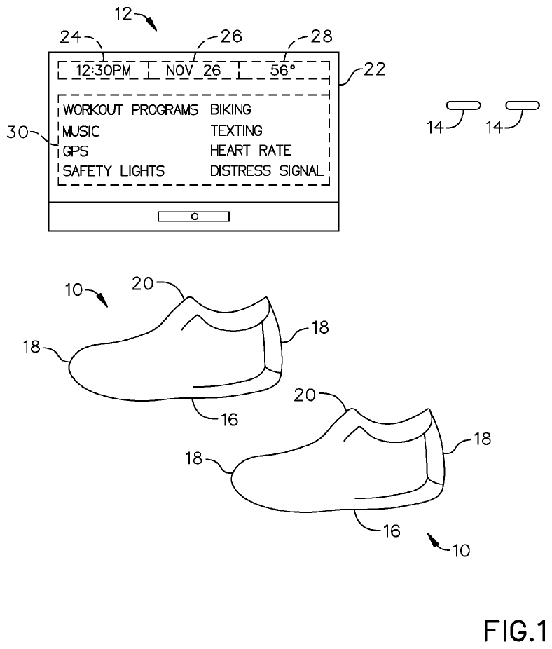

Referring to FIGS. 1 through 20, the present invention includes a workout system. The workout system includes a pair of shoes 10 and a handheld device 12. The pair of shoes 10 may include an electronic chip 16 and batteries 20. The electronic chip 16 may have an impact detector to monitor the amount of steps taken within the shoes. The electronic chip 16 may further include a wireless transceiver. The pair of shoes may further comprise of a force feedback sensor (see new Figures) that works conjointly with the light emitters. The handheld device 12 includes a processor, a wireless transceiver, a global positioning system (GPS), and a monitor 22. The transceiver of the handheld device 12 exchanges data with the transceiver of the shoes 10. The processor processes data from the transceiver and the GPS and displays the data on the monitor 22.

The processor may also transmit data indicating that a particular threshold has been met, and upon the receipt of this data the force feedback sensor, in the pair of shoes, may generate vibrational feedback to the user while turning the lights on and off. The vibrational feedback may comprise of a shake a jolt felt by the user through the heel, the middle, or front portion of the shoe. For example, a user can set a goal connected to the workout program, and upon reaching the goal the processor will send an instruction to the pair of shoes to generate the vibrational feedback. A threshold may also be indicative of a user reaching a target point in a video game. A target point may be connected to, for example, a bump on a road, shooting an item, hitting a wall, or other similarly programmable virtual reality concepts in a gaming system. A threshold may also be indicative of a target point in a movie or music video, such as certain sounds or beats. The monitor 22 may display at least one of an amount of impacts detected by the impact detector, a location of the handheld device 12, a distance traveled of the handheld device 12, and a speed of the handheld device. The present invention may include additional features.

In certain embodiments, the handheld device 12 may include a memory. The memory may store music data. In such embodiments, the system of the present invention may include an output, and headphones 14 connectable to the output. The headphones 14 may be wireless and the output may be the wireless transceiver. The handheld device 12 may further include a heart rate monitor. The shoes 10 of the present invention may further include safety lights 18.

As illustrated in FIG. 1, the monitor 22 may project a selection screen 30 in which a user may select a category of workout programs, music, GPS, safety lights, biking, texting, heart rate, distress signal, and the like. The monitor 22 may further project the time 24, the date 26 and the temperature 28. In certain embodiments, the time, distance, and other alerts may also be emitted through the headphones 14.

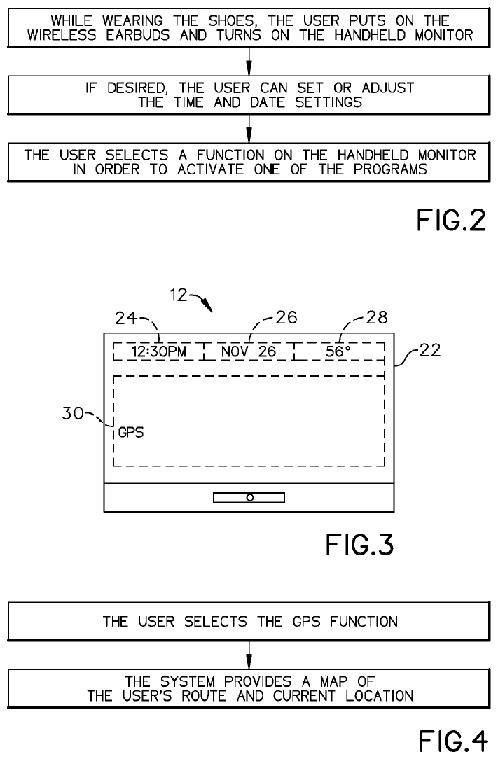

FIGS. 2 through 20 illustrate the use of the present invention. While wearing the shoes 10, the user may wear the headphones 14, such as ear buds, and turn on the handheld device 12. The headphones may comprise of The user may then select a function on the handheld monitor 12 from the selection screen 30 to activate one of the programs. As illustrated in FIG. 3, the user may select the GPS function. The monitor 22 displays a map of the user's route and current location.

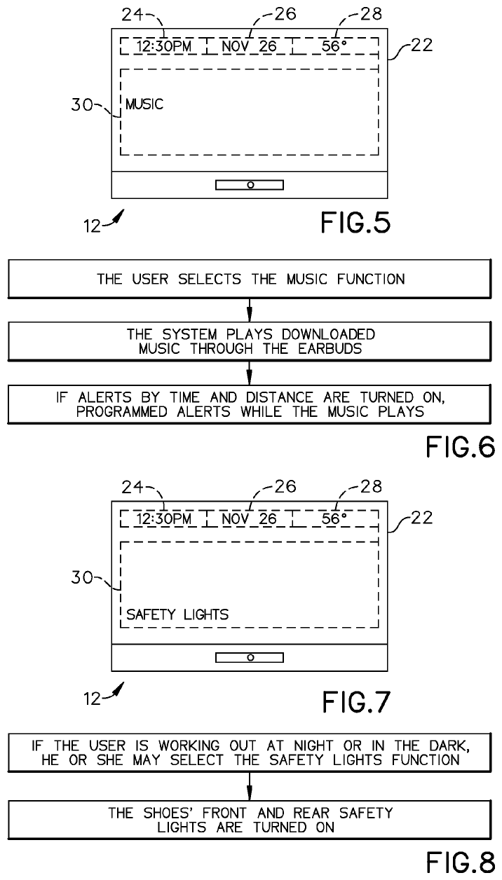

As illustrated in FIGS. 5 and 6, the user may select music from the selection screen 30. A list of music stored on the memory may be monitored for the user. The user may select the music to play on the headphones 14. Alternatively, the user may select shuffle in which the music stored on the memory may shuffle and play on the headphones 14 randomly. In certain embodiments, if the alerts are to be emitted through the headphones 14, the music volume may be lowered and the alerts may be emitted.

As illustrated in FIGS. 7 and 8, the safety lights 18 may be activated using the handheld device 12. If the user is working out at night or in the dark, the user may select the safety lights function from the selection screen 30. A command is sent from the transceiver of the handheld device to the transceiver of the shoes 10, and the safety lights 18 are turned on. The lights 18 may be located on the front and the rear of the shoes 10.

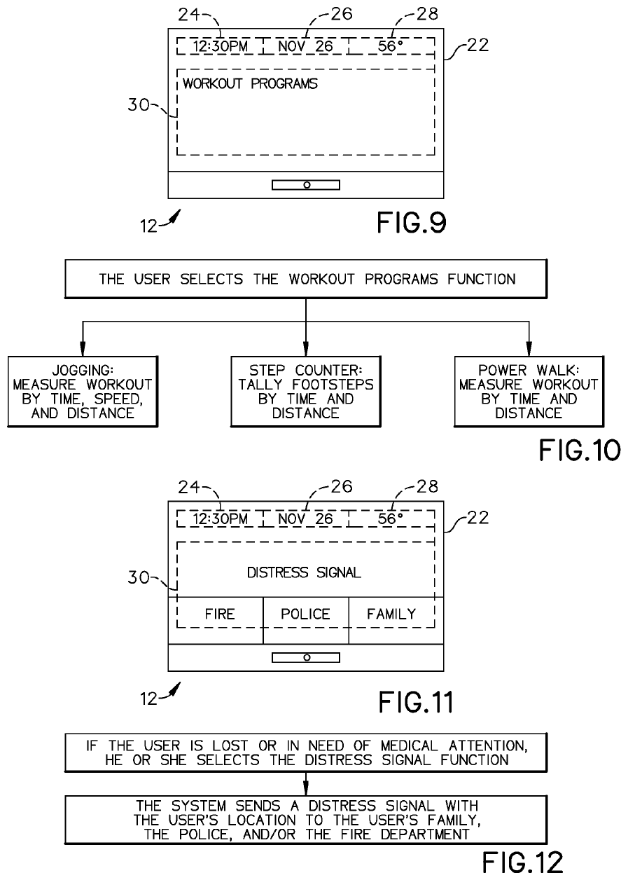

As illustrated in FIGS. 9 and 10, the user may select a workout program from the selection screen 10. Once the user has selected the workout program, the monitor 22 may project a plurality of workout routines for the user to choose from. For example, the user may select jogging, step counter, and power walk. Jogging may activate the measurement of the workout by time, speed and distance. The step counter may tally the footsteps by time and distance. The power walk may measure workout by time and distance.

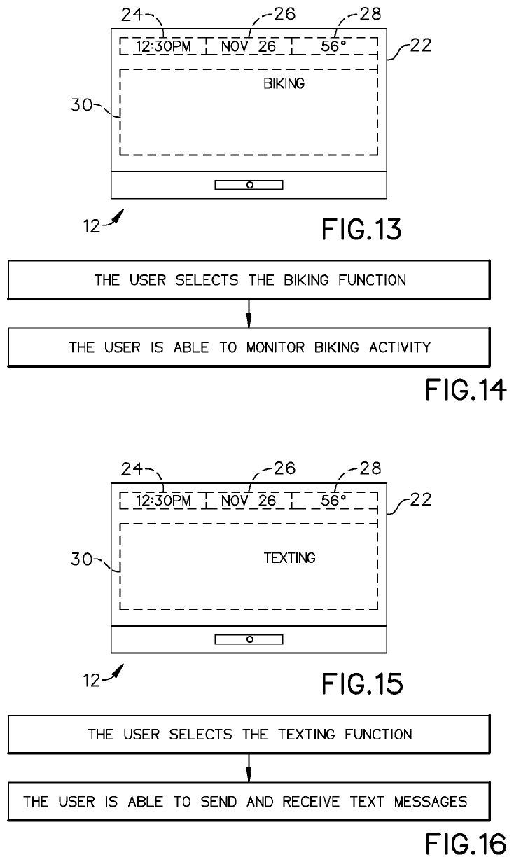

As illustrated in FIGS. 11 and 12, the user may select a distress signal from the selection screen 30. If the user is lost or in need of medical attention, the user may select the distress signal function. The user may be given the option to send the distress signal to a fire department, the police, or to family. The system sends a distress signal with the user's location to the user's family, the police and/or the fire department.

As illustrated in FIGS. 13 and 14, the user may select a biking function from the selection screen 30. In such embodiments, the user may monitor biking activity, such as speed, distance, location and the like.

As illustrated in FIGS. 15 and 16, the user may select a texting function from the selection screen 30. The user is able to send and receive text messages to other phone numbers and email addresses.

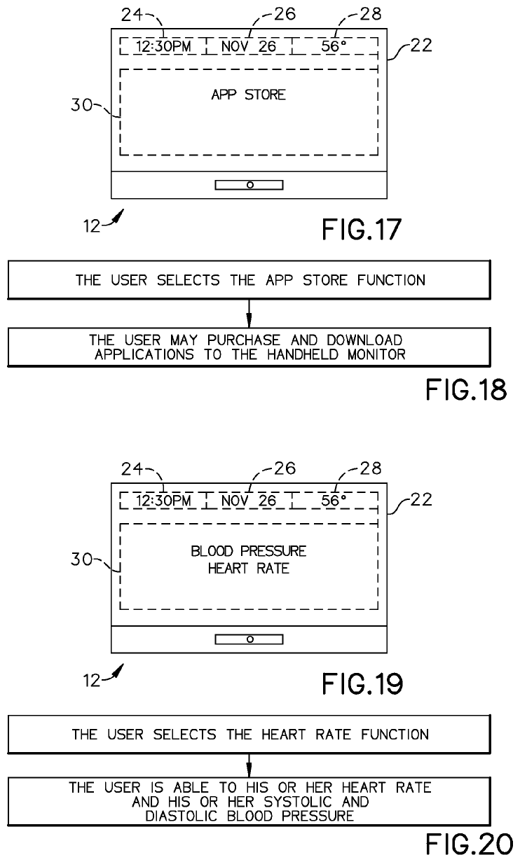

As illustrated in FIGS. 17 and 18, the user may select an app store function from the selection screen 30. In such embodiments, the user may purchase and download applications to the handheld device 10. Therefore, the handheld device 10 may include a wireless connection to the internet.

As illustrated in FIGS. 19 and 20, the user may select the heart rate function from the selection screen 30. In such embodiments, the user is able to check his or her heart rate and systolic and diastolic blood pressure. The readings may be displayed on the monitor 22.

The system may comprise of a wireless transformable game device. The wireless transformable game controller may comprise of a detachable member with a first end and a second end. The first end of the wireless transformable game device may be affixed to a first controller and the second end may be affixed to a second controller. The first and second controllers may include a force feedback sensor, an input interface on its outer surface and a speaker component on it's inner surface. The input interface may be used to transmit a set of instructions to the handheld device. The speaker component may be used to transmit sound to a user.

The processor of the handheld device may transmit data to the wireless transformable device indicating that a particular threshold has been met, and upon the receipt of this data the force feedback sensor, in the pair of shoes, may generate vibrational feedback to the user while turning the lights on and off. The vibrational feedback may comprise of a shake a jolt felt by the user through the heel, the middle, or front portion of the shoe. For example, a user can set a goal connected to the workout program, and upon reaching the goal the processor will send an instruction to the pair of shoes to generate the vibrational feedback. A threshold may also be indicative of a user reaching a target point in a video game. A target point may be connected to, for example, a bump on a road, shooting an item, hitting a wall, or other similarly programmable virtual reality concepts in a gaming system. A threshold may also be indicative of a target point in a movie or music video, such as certain sounds or beats. When in use, a user of the wireless transformable device may use the device similar to the manner in which one would use a pair of headphones. When in need, the user may detach the controllers from the detachable member and use the controller to communicate with the handheld device while using a program. The programs on the handheld device may consist of a workout program, a music program, a game program, and a video program. The video program may include music videos and movies.

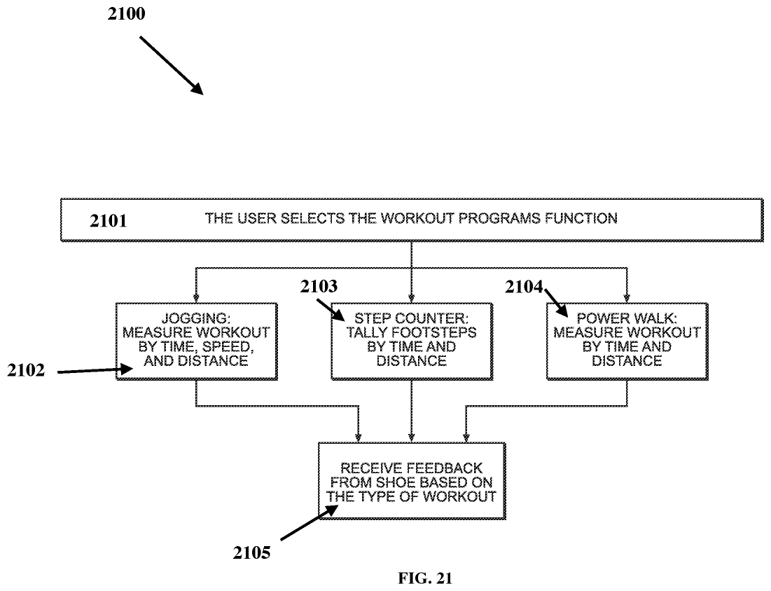

FIG. 21 is an exemplary flow chart of the force feedback function 2100. At step 2101 the user selects the workout program function. The user may select a jogging workout 2102, a step workout 2103, and a power walk 2104. Upon selection, at step 2105 feedback is received from the shoe based on the type of workout. The feedback may be force feedback, vibrational feedback, a shake, or a jolt. Thus, emitting interactive motion.

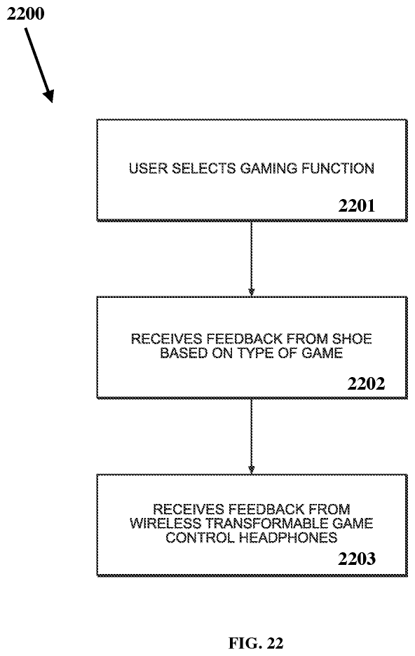

FIG. 22 is an exemplary flow chart of the force feedback function 2200. At step 2201 the user selects a gaming function. At step 2202, feedback is received from the shoe based on the type of game the user selects. At step 2203, feedback is received from the wireless transformable game controller headphones based on the game the user selects. The feedback may be force feedback, vibrational feedback, a shake, or a jolt. Thus, emitting interactive motion.

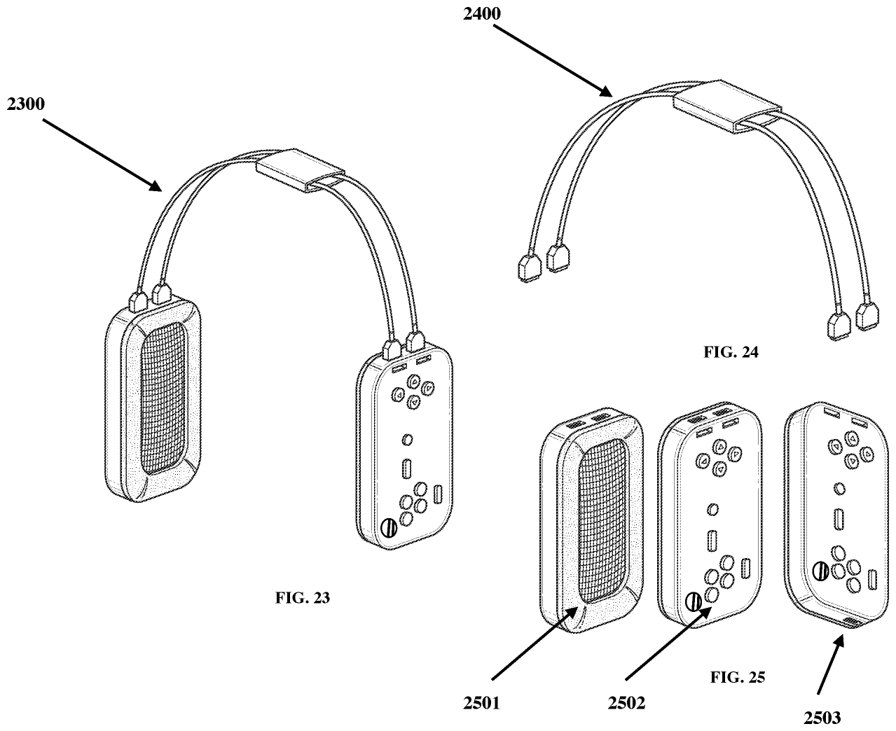

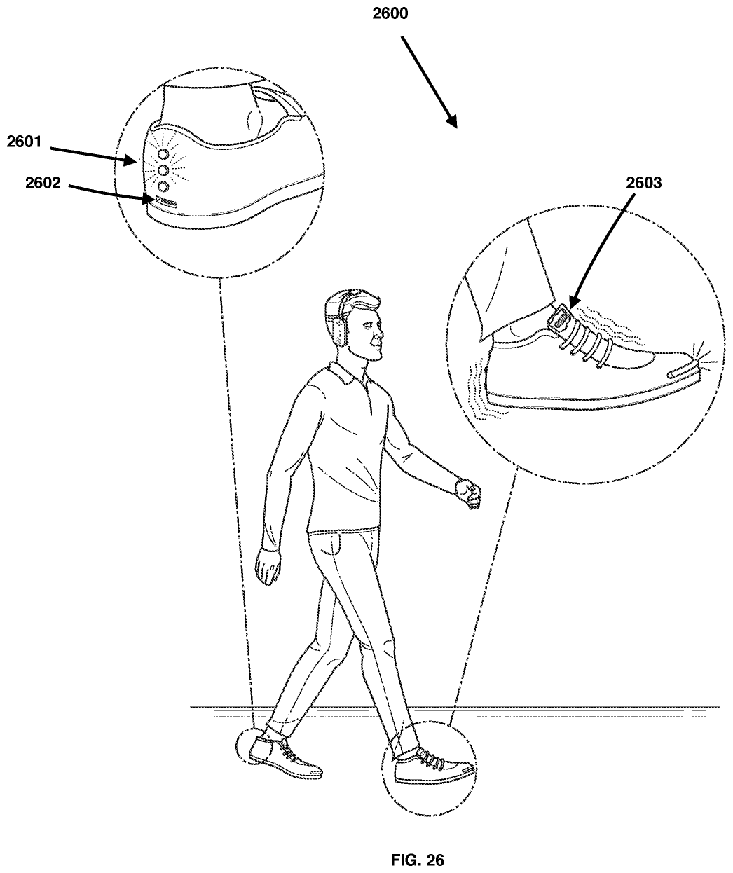

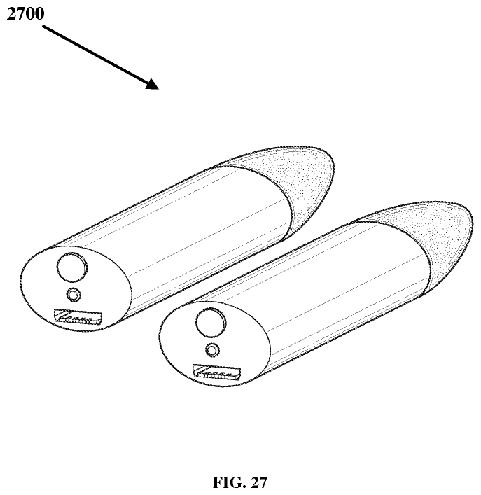

FIG. 23 is an exemplary embodiment of the wireless transformable game controller headphones. The wireless game controller headphones 2300 comprise of a detachable headband member, and two controller ear cups. The two controller ear cups comprise of a speaker, a plurality of input components, and a microphone. A user of the device may use the controller ear cups both as a listening device and as a gaming device. For example, when the controller ear cups are attached to the detachable headband member, the user may use the wireless transformable game controller as a traditional headphone. Alternatively, the user may remove the detachable headband member and use the controller ear cups as a game controller that transmits signals and data to at least one handheld device, such as the handheld device in FIG. 1. FIG. 24 is an exemplary embodiment of the detachable headband member 2400 when uncoupled to the two controller ear cups. FIG. 25 is an exemplary embodiment of the two controller ear cups 2501-2503, when uncoupled to the detachable headband member. As shown the controller ear cups comprise of a plurality of openings. A top opening provides a space to couple the detachable headband members. A bottom opening provides a space to couple a power cord. A top side opening is a charging port for the wireless earbuds, such as that in FIG. 27. The headphones also comprise of force feedback sensors. The force feedback sensors may comprise of vibrational sensors to generate a shake or jolt in response to data received from the wireless handheld device (see FIG. 1) or the pair of shoes in FIG. 26, thus emitting interactive motion.

FIG. 26 is an exemplary embodiment of the pair of shoes of the present invention. The pair of shoes comprise of a plurality of light emitting diodes at its heel 2601 and light emitting diodes at its toe 2604. The pair of shoes also comprise of a charging port 2602 at its heel and a speaker component 2603 at its tongue. Sound may emit through the speaker of the shoe based on data received from a handheld device such as the wireless transformable game controller headphones (see FIG. 23) or the wireless handheld device (see FIG. 1). As shown, the shoe also comprises of force feedback sensors. The force feedback sensors may comprise of vibrational sensors to generate a shake or jolt in response to data received from the wireless transformable game controller headphones (see FIG. 23) or the wireless handheld device (see FIG. 1), thus emitting interactive motion.

FIG. 27 is an exemplary embodiment of the wireless earbuds of the present invention. The wireless earbuds comprise of a charging port. The charging port may couple to the two controller ear cups for power input. For example, a user of the device, may use the wireless earbuds when using the two controller ear cups as a gaming device and not as a listening device. With this method, a user may use the two controller ear cups to transmit data to the handheld device in FIG. 1 and use the wireless earbuds to hear sound transmitted by the handheld device. The user may feel force feedback, vibration, and interactive motion received by the shoe and the controller ear cups during the interaction.

The computer-based data processing system and method described above is for purposes of example only and may be implemented in any type of computer system or programming processing environment, or in a computer program, alone or in conjunction with hardware.

The present invention may also be implemented in software stored on a computer-readable medium and executed as a computer program on a general purpose or special purpose pose computer. For clarity, only those aspects of the system germane to the invention are described, and product details melt known in the alt are omitted. For the same reason, the computer hardware is not described in further detail. It should thus be understood that the invention is not limited to any specific computer language, program, or computer. It is further contemplated that the present invention may be run on a stand-alone computer system, or may be run from a server computer system that can be accessed by a plurality of client computer systems interconnected over an intranet network, or that is accessible to clients over the Internet. In addition, many embodiments of the present invention have application to a various range of industries. To the extent the present application discloses a system. the method implemented by that system, as well as software stored on a computer-readable medium and executed as a computer program to perform the method on a general pail pose or special purpose computer, are within the scope of the present invention. Further, to the extent the present application discloses a method, a system of apparatuses configured to implement the method are within the scope of the present invention.

It should be understood, of course, that the foregoing relates to exemplary embodiments of the invention and that modifications may be made without departing from the spirit and scope of the invention as set forth in the following claims.

1. A system comprising:
a handheld device;
a pair of shoes;
a wireless transformable game controller; and
wireless earbuds;
wherein the handheld device comprises of a processor, a wireless transceiver, a global positioning system, a monitor, wherein the monitor comprises of a selection screen with a set of pre-determined programs;
wherein the pair of shoes comprises of an electronic chip, an impact detector, a wireless transceiver, a charging port, a speaker component disposed at the tongue of the shoe, and at least one force feedback sensor working conjointly with a light emitter, the light emitter is disposed at a front surface and a back surface of the shoe;
wherein the wireless transformable game controller comprises of a detachable member with a first end and a second end, whereby the first end is affixed to a first controller and the second end is affixed to a second controller;
wherein the wireless transceiver of the handheld device exchanges data with the wireless transceiver of the pair of shoes and whereby said data is stored in a memory database of the handheld device;
wherein the wireless transceiver of the handheld device exchanges data with the wireless transceiver of the transformable game controller;
wherein the wireless transceiver of the transformable game controller exchanges data with the wireless transceiver of the pair of shoes;
wherein the wireless transformable game controller comprises of at least two force feedback sensors, whereby said force feedback sensor is disposed within the first controller and the second controller.

2. The system of claim 1, wherein the set of pre-determined programs include a workout program, a music program, a game program, and a video program, wherein the video program may comprise of a music video and a movie.

3. The system of claim 1, wherein the first controller comprises of a set of input components, disposed within an outer surface, to wirelessly transmit a set of instructions to the handheld device, a speaker component disposed within an inner surface that transmits sound to a user, a microphone, a wireless earbud charging port, a controller charging port, and a detachable headband member; whereby the wireless earbuds connects to the wireless earbud charging port of the first controller for power input.

4. The system of claim 1, wherein the second controller comprises of a set of input components, disposed within an outer surface, to wirelessly transmit a set of instructions to the handheld device, a speaker component disposed within an inner surface that transmits sound to a user, a microphone, a wireless earbud charging port, a controller charging port, and a detachable headband member; whereby the wireless earbuds connects to the wireless earbud charging port of the second controller for power input.

5. The system of claim 1, wherein the force feedback sensors of the transformable game controller and the force feedback sensor in the pair of shoes comprises of a vibrational sensor, whereby the vibrational sensor generates a shake or a jolt in response to information received by the handheld device, emitting interactive motion; wherein the handheld device comprises of a controller operable to automatically turn the light emitter on and off as the vibrational sensor generates the shake or a jolt emitting interactive motion .

6. The system of claim 5, wherein the vibrational sensor generates the shake or a jolt in response to an instruction transmitted by the processor indicating that the system reached a pre-determined threshold.

7. The system of claim 6, wherein the pre-determined threshold comprises of a user defined threshold connected to a workout program.

8. The system of claim 6, wherein the pre-determined threshold is connected to target points in a video game.

9. The method of claim 6, wherein the pre-determined threshold is connected to target points in at least one of a video or music program.

10. A method of generating real time force feedback on a device comprising:
determining the reach of a pre-determined threshold on a handheld device;
transmitting a force feedback instruction to a user wearable device;
in response, the second device, generates a force to the user wearable device;
wherein the user wearable device comprises of a pair of shoes; wherein the pair of shoes include an electronic chip, an impact detector, a wireless transceiver, a charging port, a speaker component disposed at the tongue, and at least one force feedback sensor working conjointly with a light emitter, the light emitter is disposed at a front surface and a back surface of the shoe;
wherein the user wearable device comprises of a wireless transformable game controller, whereby the wireless transformable game controller includes a detachable member with a first end and a second end, whereby the first end is affixed to a first controller and the second end is affixed to a second controller. wherein the wireless transceiver of the handheld device exchanges data with the wireless transceiver of the pair of shoes and whereby said data is stored in a memory database of the handheld device;
wherein the wireless transceiver of the handheld device exchanges data with the wireless transceiver of the transformable game controller;
wherein the wireless transceiver of the transformable game controller exchanges data with the wireless transceiver of the pair of shoes;
wherein the wireless transformable game controller comprises of at least two force feedback sensors, whereby said force feedback sensor is disposed within the first controller and the second controller.

11. The method of claim 10, wherein the set of pre-defined programs include a workout program, a music program, a game program, and a video program, wherein the video program may comprise of at least one of a music video or a movie.

12. The method of claim 10, wherein the first controller comprises of a set of input components, disposed within an outer surface, to wirelessly transmit a set of instructions to the handheld device, a speaker component disposed within an inner surface that transmits sound to a user, a microphone, a wireless earbud charging port, a controller charging port, and a detachable headband member; whereby the wireless earbuds connects to the wireless earbud charging port of the first controller for power input.

13. The method of claim 10, wherein the second controller comprises of a set of input components, disposed within an outer surface, to wirelessly transmit a set of instructions to the handheld device, a speaker component disposed within an inner surface that transmits sound to a user, a microphone, a wireless earbud charging port, a controller charging port, and a detachable headband member; whereby the wireless earbuds connects to the wireless earbud charging port of the second controller for power input.

14. The method of claim 10, wherein the force feedback sensors of the transformable game controller and the force feedback sensor in the pair of shoes comprises of a vibrational sensor, whereby the vibrational sensor generates a shake or a jolt in response to information received by the handheld device, emitting interactive motion; wherein the handheld device comprises of a controller operable to automatically turn the light emitter on and off as the vibrational sensor generates the shake or a jolt emitting interactive motion .

15. The method of claim 14, wherein the vibrational sensor generates the shake or a jolt emitting interactive motion in response to an instruction transmitted by the processor indicating that the system reached a pre-determined threshold.

16. The method of claim 15, wherein the pre-determined threshold comprises of a user defined threshold connected to a workout program.

17. The method of claim 15, wherein the pre-determined threshold is connected to target points in a video game.

18. The method of claim 15, wherein the pre-determined threshold is connected to target points in at least one of a video or music program.

19. A non-transitory computer readable medium comprising program code when executed by a processors, is configured to cause the processor to:
determining the reach of a pre-determined threshold on a handheld device;
transmitting a force feedback instruction to a user wearable device;
in response, the second device, generates a force to the user wearable device;
wherein the user wearable device comprises of a pair of shoes; wherein the pair of shoes include an electronic chip, an impact detector, a wireless transceiver, a charging port, a speaker component disposed at the tongue, and at least one force feedback sensor working conjointly with a light emitter, the light emitter is disposed at a front surface and a back surface of the shoe;
wherein the user wearable device comprises of a wireless transformable game controller, whereby the wireless transformable game controller includes a detachable member with a first end and a second end, whereby the first end is affixed to a first controller and the second end is affixed to a second controller.
wherein the wireless transceiver of the handheld device exchanges data with the wireless transceiver of the pair of shoes and whereby said data is stored in a memory database of the handheld device;
wherein the wireless transceiver of the handheld device exchanges data with the wireless transceiver of the transformable game controller;
wherein the wireless transceiver of the transformable game controller exchanges data with the wireless transceiver of the pair of shoes;
wherein the wireless transformable game controller comprises of at least two force feedback sensors, whereby said force feedback sensor is disposed within the first controller and the second controller.

20. The method of claim 19, wherein the set of pre-determined programs include a workout program, a music program, a game program, and a video program, wherein the video program may comprise of a music video and a movie; wherein the first controller and the second controller comprise of a set of input components, disposed within an outer surface, to wirelessly transmit a set of instructions to the handheld device, a speaker component disposed within an inner surface that transmits sound to a user, a microphone, a wireless earbud charging port, a controller charging port, and a detachable headband member; whereby the wireless earbuds connects to the wireless earbud charging port of the first controller for power input; wherein the force feedback sensors of the transformable game controller and the force feedback sensor in the pair of shoes comprises of a vibrational sensor, whereby the vibrational sensor generates a shake or a jolt in response to information received by the handheld device, emitting interactive motion;
wherein the handheld device comprises of a controller operable to automatically turn the light emitter on and off as the vibrational sensor generates the shake or a jolt emitting interactive motion.
